(19) United States
(12) Patent Application Publication
(10) Pub. No.: US 2021/0228769 A1
(43) Pub. Date: Jul. 29, 2021

(54) BIOMIMETIC BIOMATERIAL AND PRODUCTION METHOD THEREOF

(71) Applicant: YILDIZ TEKNIK ÜNIVERSITESI, Istanbul (TR)

(72) Inventors: Dilek CELIK, Istanbul (TR); Cem Bulent USTUNDAG, Istanbul (TR)

(21) Appl. No.: 17/262,987

(22) PCT Filed: Jul. 25, 2019
(86) PCT No.: PCT/TR2019/050625

§ 371 Date: Jan. 25, 2021

(30) Foreign Application Priority Data
Jul. 25, 2018 (TR) 2018/10715
Jul. 24, 2019 (TR) 2019/11091

(51) Int. Cl. A61L 27/12; A61L 27/10; C01B 25/32; C08K 3/32

(52) U.S. Cl. CPC A61L 27/12; A61L 27/10; C01B 25/327; C01B 25/322; B28B 1/26; A61L 2430/02; A61L 2400/12; A61L 2400/08; C08K 2003/325; C08K 3/32

(57) ABSTRACT

This invention relates to production method comprising processes of slip casting and freeze drying, which is a hybrid system, for developing hydroxyapatite-containing bio-ceramic developed by combined utilization of medical and engineering sciences in order to use on bone diseases, wherein it discloses a new hybrid system comprising process steps of preparing a first suspension containing powder ceramic, solvent and dispersant mixture by slip casting method, molding the first suspension mixture and allowing it to dry from outside to inside, pouring excessive (residual) slip (first suspension) out of the mold when it reaches to desired thickness, removing the material shaped to form compact part (6) of the bone cortical layer from the mold, preparation of the second suspension mixture comprising powder ceramic, solvent, dispersant and binder for the formation of the trabecular part (5) by freeze drying, cooling the second suspension until the liquid (1) is frozen so as to form trabecular part (5), obtaining the solid (2) by removing the free water in the substance to be dried in the first drying phase, removing the relative water to obtain vapor (3) in the second drying phase.

### TECHNICAL FIELD

The present invention relates to production method, comprising processes of slip casting and freeze drying which is a hybrid system, of bio-ceramic biomaterial containing calcium phosphate developed by combined utilization of medical and engineering sciences in order to use on bone diseases.

### STATE OF THE ART

Bone diseases, besides causing the bone to lose its structural functions such as protection of inner body important organs and support, they also adversely affect the life quality of an individual due to reduction of homeostasis additive on calcium balance. Methods such as autograft and allograft bone transplantation are implemented as conventional treatment methods, but they cannot be utilized as effective treatment methods because of the disadvantages they have. A new treatment method at this point, tissue engineering, intends to mimic biological tissues in the best manner by using cell and bio-signal molecules. Recently, various techniques are employed in the design of tissue scaffolds to be utilized in bone tissue engineering. The present art methods are inadequate due to shortcomings of porosity, elasticity, strength and mimetic in literature.

The leading health problems lowering the life standard of human are bone defects occurred in hip, knee and other extremities. Particularly after a certain age, these problems increase by the weakening of bone structure, and bigger problems come into existence as people become unable to carry out even their daily activities. With the purpose of overcoming these problems, different methods are tried by using various materials and the works are continued. Even though selected materials become varied in the literature, adaptation to the human body, continuity of treatment, leading surgical intervention to the lowest level, not creating problems by showing toxicity-immunogenicity are top priorities in selection of materials. Hence, the above listed adverse effects are expected not to be observed in the present invention since the reason of selecting calcium phosphate-based materials and particularly hydroxyapatite are also involved in natural structure of bone. Therefore, as can be observed in the literature, calcium phosphate-based materials have been commonly preferred material for bone tissue studies. However, the cases where the techniques employed in the studies conducted with calcium phosphate-based materials remain inadequate are also encountered.

In the state of the art, US Patent application bearing the title of “Method of centrifugally slip-casting ceramic materials” with the publication No U.S. Pat. No. 2,962,790 (A) discloses slip-casting ceramic objects in water-absorbent molds. Said invention particularly relates to the art involving the centrifugal casting of hollow ceramic cylinders.

US Patent application bearing the title of “Producing a ceramic implant by coating a powder mixture of zirconia and either tricalcium phosphate or hydroxyapatite on a molded unsintered body of partially stabilized zirconia and then sintering the article” with the publication number of U.S. Pat. No. 5,185,177 (A) discloses a process for producing a ceramic implant which comprises creating a powder mixture containing alpha-tricalcium phosphate or zirconia and hydroxyapatite, in a weight ratio of 0.05 to 20. On the basis of the discovery, the invention provides a process for producing a sintered body of zirconia, which comprises subjecting partially stabilized zirconia powder to wet pulverization treatment in the presence of water and a dispersant, and slip-casting the resulting slurry. Followed by sintering, wherein as the partially stabilized zirconia powder, a powder having a BET specific surface area of from 5 to 10 m²/g is employed.

In slip casting production, it can be in powder or suspension form. Hydroxyapatite, pore agent is added as raw material, and dispersant and binder are added for intensification. Pore size and distribution are important for bone and blood vessels (must be at least 100 μm). It is advantageous in terms of pore distribution and size. The density of the hydroxyapatite material shaped by this method can be more than 96%.

Different methods used in the state of the art are described in the following.

The most common production method for calcium phosphate-based powders intended to be produced in porous form is sintering the powder mixture with hollow forming additives (such as paraffin, naphthalene, hydrogen peroxide) that evaporate at low temperatures. The pores are formed by solid state reactions in this method. However, the porosity using said technique was observed to be generally below 60%.

The replica method is the first method used for porous ceramic production. In this method, the organic porous material is saturated by the ceramic suspension which mimic the pore environment on itself via heat supply. Thus, it is ensured that the organic structure is destroyed in time and the ceramic skeleton is sintered. Porous ceramics having similar morphology with the pore structure of the initial porous material are produced. However, produced ceramic loses its mechanical properties over time because of the multiplicity of materials used.

Sacrificial phase technique is another method commonly used. It is a method consisting of two-phase ceramic composite and sacrificial phase particles. The sacrificial phase must be well dispersed in the initial ceramic powders. The homogeneous distribution of the sacrificial phase is essential, since these particles will be the pore distribution of the final porous structure.

Subsequently, this phase is removed from the mixture by means of the ways such as pyrolysis or sublimation, evaporation. The final porous structure is the negative replica of the initial sacrificial phase. This method allows for easy forming. But it takes a long time to remove the sacrificial phase particles. Furthermore, too much gas outlet during production causes environmental problems.

The direct foaming technique is based on the formation of porous structure by means of gas bubbles formed by introducing gas into the liquid medium or suspension. The material formed by this method exhibits good mechanical properties. The porosity in the resulting porous product gives information about the amount of gas added to the suspension and its pore diameter can be controlled. However, as the wet foam is an unstable system, the size of the gas bubbles can increase and the pores in the resulting product may have large diameter. Namely, stabilization of the size of gas bubbles in the wet foam is significant in this art. Although it is environment friendly and a quick method, the particles in the foam may go to rearrangement during the drying stage. This may lead to shrinkage and cracking. Thus, it requires an indirect drying method.

Paste extrusion method is a conventional technique using catalyzer, filtration to obtain honeycomb look. There are ceramic powders, mineral or polymeric binder additives and lubricating agents inside the paste. Then, the end product is formed by drying, sintering steps as is in classical ceramic production process by mechanical extrusion. The most advantageous point of it is that formation well defined end product can be provided by creating standard production in desired forms. This creates a good option for specific applications. However, porous ceramic production by use of too much lubricating agent and polymeric binder may bring along environmental problems.

Rapid prototyping and 3D printing tech are useful methods developed for the production of three-dimensional complicated prototypes. Stereolithography (SLA), Selective Laser Sintering (SLS), Laminated Object Manufacturing (LOM), Fused Deposition Modeling (FDM), Solid Ground Curing (SGC) are commercially available ones. All rapid prototyping techniques are based on the principle of creation of three-dimensional structure layer by layer. Three-dimensional structure is formed with a continuous filament from the nozzle tip ranging from 50 μm to 1 mm under the control of computer. It is a technique allowing formation of complicated shapes compared to other techniques and being more controlled. Its only disadvantage is its high price.

Most of these methods used for porous ceramics starts from colloidal ceramic suspensions. Well defined and stabile ceramic suspensions are required for these production techniques. When ceramic components are prepared, the colloidal production is performed in five steps: the first step is powder synthesis, the second step is suspension preparation, the third step is formation of desired shape, the fourth step is removal of solvent phase, the fifth step is condensation. The characterization difference in colloidal systems is the extent of the interaction between the particles and the dispersion medium. The behavior or structure of suspension is highly influenced by interparticle forces or surface forces. There are several ways to obtain hydroxyapatite ceramics by colloidal systems, such as freeze casting, gel casting, tape casting, slip casting.

High porosity and compressive strength are emphasized in the literature for the end products obtained by freeze casting method. It is focused on porous ceramic production and its mechanical properties in the recent researches. Finding out the associations between rheologic properties of initial suspension, characteristic of resulting porous structure and the mechanism of freezing process is little if any inadequate.

Tape casting is a technique by which ceramic can be produced in very thin and flat form. It is advantageous that it forms asperities when the organic binders evaporate. However, it is a disadvantage that it requires evaporation at high temperature as it damages the resulting product quality.

In the Gel casting method, asynchronous solidification may take place for various reasons such as the temperature gradient in the suspension, initiator distribution, etc. This may cause cracking in the end product. It may create problem in homogenization.

### DESCRIPTION OF THE INVENTION

This invention is intended to develop hydroxyapatite bio-ceramics by using the method defined as hybrid system and the slip casting method and freeze-drying methods together. It has been determined that the production methods of produced materials to function similarly in the state of the art is not sufficient to have the properties altogether such as porosity close to natural one as expected from the material, imitating bone form as very dense at outside and as trabecular at inside, having sufficient robustness for the area carrying load, presence of inner pore connections to which bone cells can hold, not being so brittle. The design of the hydroxyapatite material is planned to be completed by making intense outer part of bone by slip casting and spongy inner part by freeze drying method. Bone marrow cavity will be provided in the inner middle part by the appropriate mold design. Thus, the compensation of the existing deficiencies is aimed. Furthermore, another object of the invention is to overcome above listed problems.

The main reason why the materials produced in the state of the art do not have the desired properties altogether lies in the fact that the production method compatible with the material used is not preferred or cannot be found. The material best imitating the natural bone structure can be made by ensuring the formation of compact part imitating dense and hard outer layer of bone tissue by slip casting method and formation of trabecular (porous) layer under the compact part by freeze drying method in the developed production method. Use of porous hydroxyapatite bio-ceramics produced by this manner on bone tissue will be appropriate in areas carrying load mechanically. Thus, the problem of restricted area of use in the body encountered till now and lead to new composite material will be eliminated. Retention, growth and reproduction of bone cells by the fact that inner pore connections are more advanced will make the material to be accepted by the body easier. By the combination of these two methods, a structure and material imitating bone tissue has been developed.

### DESCRIPTION OF THE FIGURES

FIG. 1: illustrates the schematic representation of the slip casting method

FIG. 2: illustrates schematic representation of the freeze-drying method

FIG. 3: illustrates schematic representation of the biomimetic material produced

FIG. 4: illustrates the sintering temperature-time graphic

FIG. 5: illustrates the Hydroxyapatite FT-IR analysis graphic

FIG. 6: illustrates the hydroxyapatite particle size distribution analysis

FIG. 7: illustrates the hydroxyapatite XRD Analysis

FIG. 8: illustrates the PVA thermogravimetric diagram

FIG. 9: illustrates the SEM image of surface of hybrid tissue scaffold

FIG. 10: illustrates the pore distribution graphic of surface of hybrid tissue scaffold

FIG. 11: illustrates the SEM image of hybrid tissue scaffold with magnification of 50.23K X

FIG. 12: illustrates particle size distribution graphic of hybrid tissue scaffold at magnification of 50.23K X

### DESCRIPTION OF REFERENCE NUMBERS

| No | Description |
|---|---|
| 1 | Liquid |
| 2 | Solid |
| 3 | Vapor |
| 4 | Bone Marrow Cavity |
| 5 | Trabecular Part |
| 6 | Compact Part |

### Detailed Description of the Invention

The process used in the present invention is a hybrid system unlike the methods available in the state of the art. The novelty of this invention is the development of a new technique and biomaterial using slip casting and freeze-drying methods together. Thus, the formation of bone model having the best biomimetic properties will be provided.

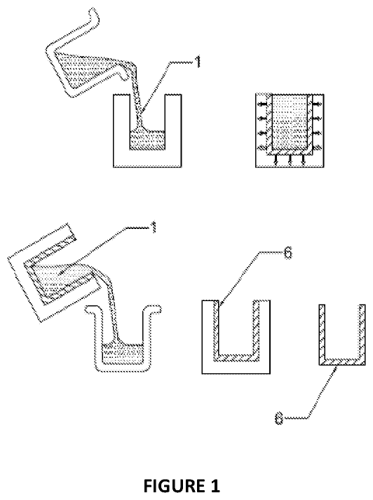

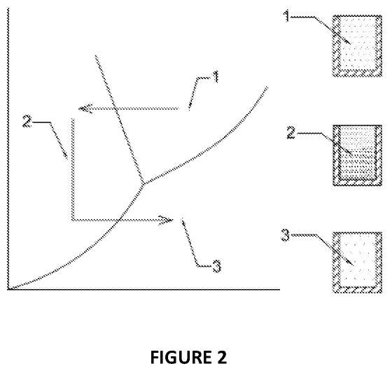

The process steps of the production of biomimetic biomaterials comprise the following steps:
Preparation of the first suspension which is an essential step for the slip preparation; is prepared with the powder ceramic, solvent and dispersant mixture that are the main construction elements.
The prepared slip bone compact part (6) is the first suspension to be used as mimetic and having a certain viscosity to be molded in slip casting technique application.
For designing the segment mimetic of the bone compact part (6), the technique which is based on leaving the slip for drying from out to inside after molding and removing excessive (residual) slip from mold when it reaches desired thickness is used. The portion that remain unremoved will function as the compact part (6) in the bone tissue scaffold.
For designing the segment mimetic of the bone trabecular part (5); primarily the second suspension mixture containing powdered ceramic, solvent, dispersant and binder is prepared.
The compact part (6) formed in the slip mold will be formed in the inner part. In use as tissue scaffold in bone tissue engineering, retention, proliferation, migration, nutrient and oxygen permeability, vascularization of the cells will be ensured. In the mold design, the innermost portion will be the bone marrow cavity (4).
In slip casting method; the first suspension (slip) is casted into the mold, the water in the suspension is absorbed by the porous mold, the excess suspension is removed, and the formed material is taken out from the mold. Thus, bone cortical layer is formed. (FIG. 1)
Freeze drying method is considered to be a good method for improving the long-term stabilization of colloidal nanoparticles. The end product having the best quality is obtained by this method when compared to other methods. The most important factor is that the surface in which sublimation is occurred by means of structural hardness is realized by the fact that it is frozen. The material form is not deformed also after drying process. It consists of three phases; 1) freezing, 2) first drying, 3) second drying. In the freezing phase, the suspension (slip) liquid (1) is cooled until it freezes. In the first drying phase, solid (2) is obtained by removing the free water in the material to be dried and, in the second freezing phase, steam (3) is obtained by removing relative water. (FIG. 2)

By the combination of these two methods, a structure mimicking the bone tissue will be developed.

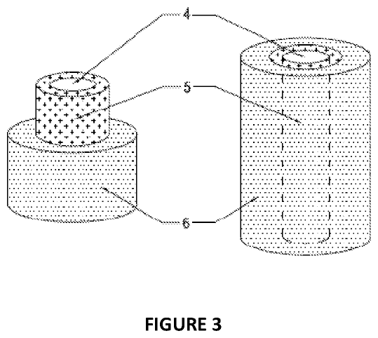

In the exemplary embodiment of the subject matter product shown in FIG. 3, the bone marrow cavity (4), the trabecular part (5) obtained by freeze drying and the compact part (6) obtained by slip casting constitute the biomimetic material.

An embodiment of the inventive production method comprises the following process steps:
By means of the slip casting method,
   preparation of the first suspension mixture comprising powder ceramic, solvent and dispersant,
   molding the first suspension mixture and allowing it to dry from outside to inside,
   pouring excessive (residual) slip (first suspension) out of the mold when it reaches to desired thickness,
   removing the material shaped to form compact part (6) of the bone cortical layer from the mold,
By means of the freeze-drying method,
   preparation of the second suspension mixture comprising powder ceramic, solvent, dispersant and binder,
   cooling the second suspension until the liquid (1) is frozen so as to form trabecular part (5),
   obtaining the solid (2) by removing the free water in the substance to be dried in the first drying phase,
   removing the relative water to obtain vapor (3) in the second drying phase.

A further significant characterization of the inventive production method comprising the slip casting and freeze drying processes which is a hybrid system for developing bio-ceramic is that it comprises the process step of preparing the first suspension containing the mixture comprising powder ceramic in the ratio of 40%-70% by weight, solvent in the ratio of 30%-60%, dispersant in the ratio of 0.1%-1% by weight of powder ceramic. Furthermore, it also comprises the process step of preparing the second suspension containing the mixture comprising powder ceramic in the ratio of 40%-70% by weight, solvent in the ratio of 30%-60%, dispersant in the ratio of 0.1%-10% by weight of powder ceramic, binder in the ratio of 1%-10% by weight of powder ceramic.

The first suspension used to form the bone cortical layer, the compact part (6) by the slip casting method comprises powder ceramic, solvent, dispersant, and the second suspension used to form the trabecular part (5) by freeze drying method is obtained by mixing powder ceramic, solvent, dispersant and the binder. In the present invention, preferably, calcium phosphate as powder ceramic; water and/or organic solvent(s) as solvent; stabilizer(s), surfactant(s) and/or antifoam(s) are used as dispersants. In the invention, sodium tripolyphosphate and/or ammonium polyactylates as dispersants, polyvinyl alcohol (PVA) and/or carboxy methyl cellulose (CMC) are preferably used as binder.

What is more, the subject matter production method is a method applicable to industry. Consistency of the product and production method is ensured by means of the different technical effect shown by combining the two methods to be used as hybrid. This developed system also has the property to be produced industrially by designing the appropriate production processes.

What is more, the subject matter production method also comprises hydroxyapatite synthesis. Hydroxyapatite is a calcium phosphate-based bio-ceramic. In the production method, hydroxyapatite (powder ceramic) synthesis has been performed by the wet chemical precipitation using orthophosphoric acid (H₃PO₄) and calcium hydroxide (Ca(OH)₂) chemicals. In one embodiment of the present invention, the suspension is prepared with a mixture of orthophosphoric acid of 80-85% and calcium hydroxide. A further characterization of the production method is; nano-sized hydroxyapatite (powder ceramic) synthesizing by the wet chemical precipitation method. The prepared hydroxyapatite (calcium phosphate) will be used as the powdered ceramic in the ratio of 40-70% which constitutes the mixture in the process step of preparing the suspensions (first and second suspensions) in the subject matter production method.

The inventor has carried out works to observe the technical effect of the subject matter production method. During the works, the synthesized hydroxyapatite by the wet chemical precipitation method in nano-size and obtained the product as result of the subject matter production method by using slip casting and freeze-drying techniques on shaping as hybrid.

Wet chemical precipitation method was used in the production of powder hydroxyapatite. Moreover, the hybrid shaping method used in the literature for the very first time is the combined application of slip casting and freeze-drying techniques. The slip casting technique makes it possible to obtain reliable ceramic bodies at high density values and is used to mimic the bone compact part (6). Since pore structures connected to each other three dimensionally and being well defined by the freeze-drying method, it is used to mimic trabecular part (5). In the characterization of the scaffolds obtained FTIR, DLS, XRD, TG-DTA, BET, He Pycnometer and SEM analysis methods were used.

A summary of the work carried out by the inventor is given below. In the experimental work, primarily hydroxyapatite synthesis was realized. Then, polyvinyl alcohol solution was prepared, mold design and production were conducted. Subsequently, slip casting and freeze-drying process steps were applied and ended with sintering process.

Firstly, in hydroxyapatite synthesis, suspension was prepared with the mixture of orthophosphoric acid (H₃PO₄) of 80-85% and calcium hydroxide (Ca(OH)₂). 0.6 M (20.47 ml) orthophosphoric acid was weighed, and distilled water was added up to 500 ml. 0.5 mol (37.045 g) of calcium hydroxide (Ca(OH)₂) was weighed. The orthophosphoric acid was put in the magnetic stirrer and, during stirring at medium speed (450 rpm), calcium hydroxide was added to the acid-water mixture by means of a spatula. The addition process was continued until the calcium hydroxide was completely ran out.

When the addition was completed, the mixture had a white homogeneous appearance. And then, mixed by adjusting the pH level.

6 tubes of 35 ml were centrifuged at 3000 rpm for 10 min at the washing process. They were placed in the centrifuge after vortexing. Then, synthesized hydroxyapatite within centrifuge tubes were left for drying. These steps were repeated twice for wet chemical precipitation. As result of this process, a total of 600.87 g of powder hydroxyapatite was obtained.

Polyvinyl alcohol solution was prepared as the secondary step. In hydroxyapatite-based bio-ceramics, PVA has been preferred to eliminate brittleness and to prolong material life by providing mechanical stabilization. 10.5 g of polyvinyl alcohol was weighed and dissolved in 200 ml of distilled water. In order to carry out dissolution, it was mixed in magnetic mixer at 85° C. degree for 4 hours. When the reaction was completed, a homogeneous PVA solution was obtained.

Two materials, as silicone and gypsum, were used for design and production of the mold.

In the processes of slip casting and freeze drying, while polyvinyl alcohol (PVA) was used as binder to make the synthesized hydroxyapatite powder form into slip, sodium tripolyphosphate (STPP) surfactant was used as dispersant.

Different hydroxyapatite-bearing suspensions were prepared to mimic the compact part (6) and trabecular part (5) of the bone for slip. For the compact part (6), casting is performed by preparing three different hydroxyapatite-bearing suspensions, which are respectively 28.5%, 37.5% and 50%, containing 20 g, 30 g, 50 g of hydroxyapatite in 50 ml of distilled water and wherein STPP at the amount of 0.32% of the powder ceramic (hydroxyapatite) was added to each suspension. For the trabecular part (5); suspension was prepared by using 30 ml of distilled water, 20 g of hydroxyapatite, 2 g (40 ml) of PVA (10% of hydroxyapatite), STPP at the amount of 10% of hydroxyapatite. The suspension containing 50% powder ceramic (hydroxyapatite) among the three different suspensions, showed optimum retention to mold and drying behavior.

Slip casting and freeze-drying processes performed separately were applied as hybrid. As so in the natural bone structure, the compact part (6) surrounding the spongy layer was made by slip casting primarily with the first suspension of hydroxyapatite+distilled water+STPP in order to obtain the mimetic structure. The material that was not dried after the first suspension was dried until it reached to a certain thickness around the gypsum mold was discharged back. The second suspension Hydroxyapatite+PVA+distilled water+STPP for the trabecular part (5) was cast into the gypsum mold, which was previously slip casted and the middle part of which was hallow. After the castings were completed, freeze drying technique was applied by lyophilizer. Ceramic molds were used for slip casting and freeze-drying processes performed. Silicone molds were cracked during retention at −80° C. for freeze drying process and they were considered to be inappropriate for this assembly.

During the sintering process step, the chemicals used in the wet chemical precipitation method to synthesize hydroxyapatite affect the sizes of formed hydroxyapatite particles. Hydroxyapatite synthesized with orthophosphoric acid and calcium hydroxide has larger particle sizes compared to hydroxyapatite particle sizes synthesized with calcium nitrate tetrahydrate and diammonium hydrogen phosphate. This increases the temperature degree required for sintering.

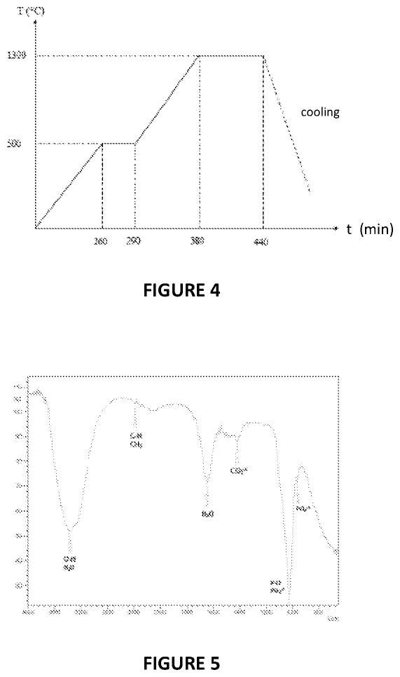

Firing was carried out at different temperatures as 900° C. and 1200° C. for the prepared hybrid hydroxyapatite tissue scaffolds, but it was observed that the sintering was not realized as expected. The optimum sintering temperature for the hybrid hydroxyapatite tissue scaffold was determined as 1300° C. (FIG. 4)

The inadequacy of the surface porosity of tissue scaffolds obtained by merely applying slip casting and the brittleness problem of tissue scaffolds obtained by merely applying freeze drying were overcome by the use of these two techniques. Thus, the disadvantages, indicated in the literature, such as being inadequate for load carrying bones, have been eliminated.

Hybrid shaping methods to be applied for the first time in the literature within the scope of developed production art are; the slip casting and the freeze-drying techniques. Slip casting technique provides higher density values and therefore ensures obtaining more reliable ceramic bodies. By means of the three-dimensional hybrid design, layers having a structure similar to natural bone will be formed. While high densities cause a brittle structure in other works, brittleness will be avoided as they are supported to form a spongiosis layer by the freeze-drying technique. Suitable environment will also be formed for the cells to retain. Furthermore, since the bone marrow cavity (4) is also formed by specific mold design as is natural bone form, mechanic resistance will also be appropriate for load carrying bones in the body.

In the characterization of the scaffolds obtained FTIR, DLS, XRD, TG-DTA, BET, He Pycnometer and SEM analysis methods were used.

Analysis was carried out in the range of 4000-400 cm⁻¹ for HA powders synthesized by wet chemical method. HA particle measurements synthesized with orthophosphoric acid and calcium hydroxide chemicals are shown. (FIG. 5) Shows in general 3000-2800 cm⁻¹ CH₃ group (methyl) C—H bonds, 3300 cm⁻¹-3600 cm⁻¹ characteristic —OH ion band, 1200-1600 cm⁻¹ CO₃⁻² band (carbonate ions), 1000-1100 cm⁻¹ PO₄⁻³ existence.

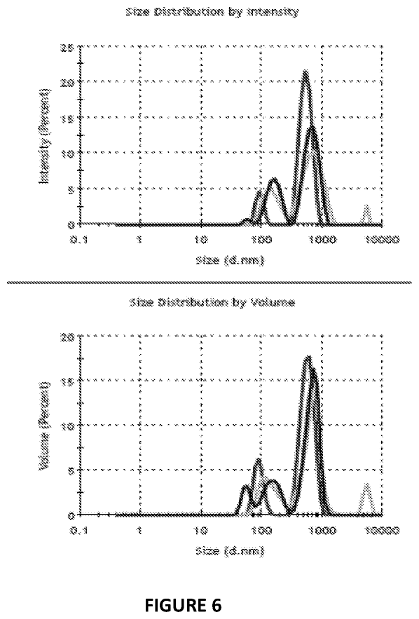

A particle size distribution analysis for HA was performed and the resulting graphic is shown in FIG. 6. The mean particle size for HA was measured as 137.8 nm and the PDI value as 0.59. According to the DLS results, the irregular distribution in the graphics shows the existence of regional agglomeration.

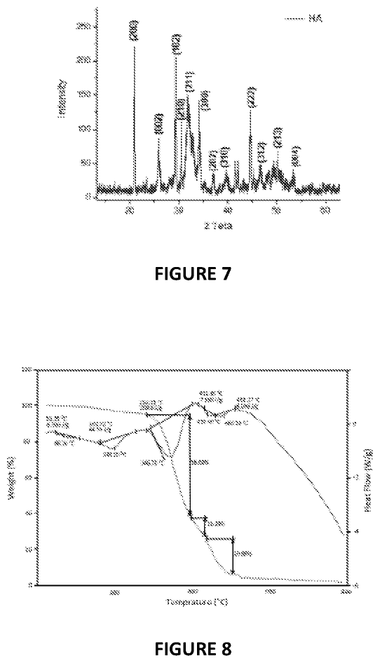

In order to characterize the phase composition and crystallinity of HA, XRD which is a routinely used analysis was utilized. XRD analysis of HA is shown in FIG. 7. In the XRD patterns of the material, increasing peaks around the planes (002), (211), (300), (202), (222), (310), (213) and (004) are observed.

(PVA) thermal analysis of poly(vinylalcohol) used as binder is shown in FIG. 8.

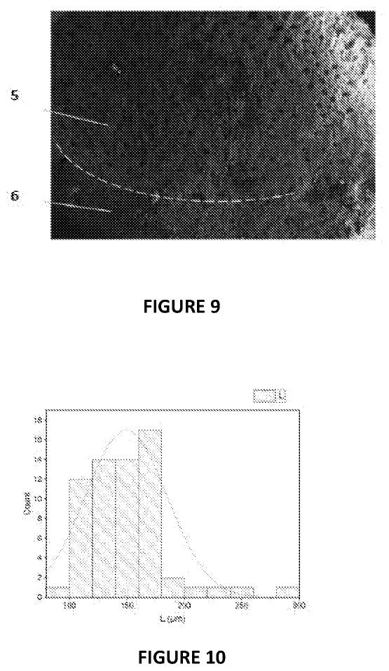

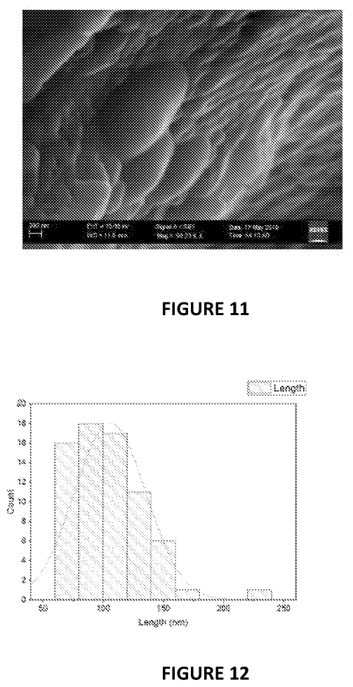

SEM image of tissue scaffold prepared as hybrid is shown in FIG. 9. It is clearly seen in this image that the cortical layer—the compact part (6), formation of which is expected to be done by the slip casting, and the spongy layer—the trabecular part (5), formation of which is expected to be done by freeze drying, are formed. The pore distribution graphic of the SEM image is shown in FIG. 10.

SEM image of hybrid tissue scaffold with magnification of 50.23K X is seen in FIG. 2. The particle size distribution graph of said image is shown in FIG. 12.

The specific surface area of the HA powder was measured to be 55.11 m²/g. Estimated equivalent particle size was computed as 34.5 nm. The theoretical density of hydroxyapatite is 3.16 g/cm³. Particle size measurement at smaller sizes are obtained when the particle sizes (34.5 nm) obtained by BET analysis are compared to DLS analysis (137.5). The reason of that can be agglomeration occurred in the HA suspension prepared for DLS measurement (Gervaso et al., 2012). According to XRD analysis lattice tendency, average value (46 nm) of particle size conducted with Scherrer equation supports the BET analysis result (34.5 nm).

He pycnometer was used to calculate the density of bio-ceramic tissue scaffold developed as hybrid. The mass was measured as 3.4694 g, the volume as 1.1032 cm³ and the density was calculated as 3.1450 g/cm³ for the hybrid tissue scaffold. This result for hybrid tissue scaffold shows that this tissue scaffold which was developed by expecting that it has bone tissue mimetic has a porous structure.

The bulk density value of the hybrid tissue scaffold was calculated as 2.19 g/cm³ and the bulk volume value as 1.579 cm³. When calculating the bulk density and volume, the standard deviation was found to be 0.1 as result of the radius (0.72 cm) and the height (0.97 cm) taken from different regions of the hybrid tissue scaffold.

While performing nutrient and waste diffusion, it is proved that tissue scaffold that can also support cell proliferation and vascularization is developed by means of the existence of micro and nano-sized pores obtained by the images. Furthermore, it is seen that the average particle size is 100 nm. Depending on the sintering process, it is also observed that merging occurs on the HA particles. The mechanical resistance of the tissue scaffold will be supported by these mergers.

HA particle size of hybrid tissue scaffold is 50-120 nm and the surface porosity values are in the range of 100-180 μm. These values indicate that the inner pore connections essential for vascularization have appropriate sizes. Distribution of pores at micro and nano-sizes proves that an appropriate tissue scaffold for nutrient-gas diffusion and waste elimination has been developed.

In general, it is concluded that a hybrid tissue scaffold has been developed, wherein vascularization can be achieved by the presence of appropriate sized surface porosity and internal pore connections as expected from the bone tissue scaffold; and which can support cell retention, migration and proliferation; and wherein the basic requirements such as nutrient diffusion and waste elimination are met.

1. (canceled)

2. A bioceramic hybrid system production method comprising hydroxyapatite used on bone disease, the method comprising:
utilizing a slip casting method to:
prepare a first suspension mixture comprising powder ceramic, solvent and dispersant,
pouring into a silicon or gypsum mold the first suspension mixture and allowing it to dry from outside to inside,
pour excessive (residual) slip (first suspension) out of the mold when it reaches to desired thickness,
remove the material shaped to form compact part (6) of the bone cortical layer from the mold, and
utilizing a freeze-drying method to:
prepare a second suspension mixture comprising powder ceramic, solvent, dispersant and binder,
pour the second suspension into the slip casted first suspension (compact part (6)),
cool the second suspension until the liquid (1) is frozen so as to form trabecular part (5),
obtain the solid (2) by removing the free water in the substance to be dried in the first drying phase, and
remove the relative water to obtain vapor (3) in the second drying phase,
wherein the method further comprises a process step of preparing the first and the second suspensions comprising the mixture comprising powder nano-sized ceramic (hydroxyapatite) synthesized by the wet chemical precipitation method using orthophosphoric acid (H₃PO₄) of 80%-85% by concentration and calcium hydroxide (Ca(OH)₂) chemicals.

3. The production method of hydroxyapatite-containing bio-ceramic according to claim 2, wherein the method further comprises the process step of preparing the first suspension containing the mixture comprising powder ceramic in the ratio of 40%-70% by weight, solvent in the ratio of 30%-60%, dispersant in the ratio of 0.1%-1% by weight of powder ceramic.

4. The production method of hydroxyapatite-containing bio-ceramic according to claim 3, wherein the method further comprises the process step of preparing the first suspension containing the mixture comprising powder ceramic in the ratio of 50% by weight.

5. The production method of hydroxyapatite-containing bio-ceramic according to claim 2, wherein the method further comprises the process step of preparing the second suspension containing the mixture comprising powder ceramic in the ratio of 40%-70% by weight, solvent in the ratio of 30%-60%, dispersant in the ratio of 0.1%-10% by weight of powder ceramic, binder in the ratio of 1%-10% by weight of powder ceramic.

6. The production method of hydroxyapatite-containing bio-ceramic according to claim 2, wherein the method further comprises the process step of preparing the first suspension mixture comprising the mixture of calcium phosphate (hydroxyapatite) as powder ceramic, water and/or organic solvents as solvent, and sodium tripolyphosphate and/or ammonium polyacrylate as dispersant.

7. The production method of hydroxyapatite-containing bio-ceramic according to claim 2, wherein the method further comprises the process step of preparing the second suspension mixture comprising the mixture of calcium phosphate (hydroxyapatite) as powder ceramic, water and/or organic solvents as solvent, sodium tripolyphosphate and/or ammonium polyacrylate as dispersant, and polyvinyl alcohol (PVA) and/or carboxy methyl cellulose (CMC) as binder.

8. (canceled)

9. The production method of hydroxyapatite-containing bio-ceramic according to claim 2, wherein the method further comprises the process step of preparing the suspensions comprising the mixture comprising powder ceramic hydroxyapatite sintered at 1300° C. for tissue scaffold after synthesized by the wet chemical precipitation method.
